(19) United States
(12) Patent Application Publication
(10) Pub. No.: US 2011/0071119 A1
(43) Pub. Date: Mar. 24, 2011

(54) HEAT RESISTANT DELIVERY SYSTEM

(75) Inventor: Judy Davis, San Clemente, CA (US)

(73) Assignee: Hero Nutritionals, LLC, San Clemente, CA (US)

(21) Appl. No.: 12/779,322

(22) Filed: May 13, 2010

Related U.S. Application Data
(60) Provisional application No. 61/243,641, filed on Sep. 18, 2009.

(51) Int. Cl. A61K 31/616; A23G 3/36; A23L 1/302; A23L 1/304; A23L 1/30; A23L 1/0532; A23L 1/0522; A23L 1/0524; A23L 1/0562

(52) U.S. Cl. 514/165; 426/573; 426/72; 426/74; 426/61; 426/575; 426/578; 426/577; 426/576

(57) ABSTRACT

A heat resistant chewable composition for the oral delivery of dietary supplements and pharmaceutical compounds. The chewable composition includes a delivery vehicle and an active ingredient incorporated therein. The delivery vehicle may include a gummy candy made from a binding agent with thermo-irreversible characteristics. The active ingredient may include an over-the-counter drug or a prescription drug to provide a desired effect on the user. The active ingredient may also include any combination of nutraceuticals, vitamins, minerals, antioxidants, soluble and insoluble fiber, herbs, plants, amino acids, probiotics, prebiotics, fatty acids, digestive enzymes, or any other health promoting ingredient.

### RELATED APPLICATIONS

This application claims priority from U.S. Provisional Application No. 61/243,641, filed on Sep. 18, 2009, titled HEAT RESISTANT DELIVERY SYSTEM, which application is incorporated in its entirety by reference in this application.

### BACKGROUND OF THE INVENTION

1. Field of the Invention

The invention relates generally to a heat resistant delivery system, and more particularly, to an edible thereto-irreversible composition for the oral delivery of nutritional supplements and pharmaceutical compounds, and a method for manufacturing the same.

2. Related Art

Recently, chewable dietary supplements have been manufactured and sold in the form of gummy (also sometimes spelled as “gummi”) candy supplements. Now a selection of health supplements are being manufactured and sold in a chewable gummy form, including both children and adult supplements. The introduction of gummy supplements into the marketplace has been particularly helpful in getting children to take daily vitamin supplements. For adults that do not like swallowing pills, gummy supplements have also provided a non-pill alternative for adults to get their daily vitamin requirements.

Although gummy candy was first introduced in 1920 as “gummy bears,” it was not until very recently that gummy candy was utilized as a delivery system for supplements. Traditional gummy candy is made from a gelatin base, which is similar to the base found in soft caramels, marshmallows, foam-filled wafers, licorice, wine gums, pastilles, chocolate coated mallows and a host of other sweets. Gelatin is a protein derived from animal tissue that forms thick solutions or gels when placed in water. Gelatin serves as a binding agent that gives the candy its elasticity and the desired chewy and rubbery consistency.

Because gelatin is a tasteless and odorless compound, sweeteners and flavorings are typically added to the gelatin base to give the gummy candy its taste. Thus, in addition to gelatin, gummy candies are generally made from a blend of water, sweeteners (e.g., corn starch, corn syrup, and/or sucrose), flavors, and colors.

When mass produced, gummy candies are made from a gelatin base or stock that's mixed and pumped into a special candy cooker that cooks the gelatin base by combination of pressure and steam. Once cooked, a vacuum is applied to the cooked candy to remove excess moisture therefrom. The cooked candy then moves to a mixing station where colors, flavors, and food grade acids are mixed into the gelatin base. Next, a starch molding machine, commonly known as a mogul, pumps the cooked gelatin stock into starch-filled mold boards that shape the individual candies. After curing, the gummies are removed from the molds and then packaged, delivered, and sold.

The textural characteristics of gummy candies depend on many factors, such as temperature, method of manufacture, and pH. Because gelatins are thereto-reversible, which means they get softer and thinner as they are heated, gelatin-based candies have a tendency to melt when they are exposed to high temperatures during storage and transport. Therefore, gelatin-based delivery systems, more particularly, traditional gummy candies, have a relatively short shelf life in high temperatures and have a tendency to melt during transport to far away locations.

Thus, a need exists for a chewable delivery system suitable for administering health supplements that will retain its shape and textural characteristics when exposed to high temperatures during storage and transport.

### SUMMARY

A heat-resistant delivery system for delivering nutritional supplements and pharmaceuticals is provided. The delivery system generally includes a heat resistant delivery vehicle in the form of an organic or non-organic candy, and a predetermined dosage of an active ingredient, in liquid or powered form. By ingesting the candy, the consumer is able to directly supply its body with the active ingredient.

According to one implementation, the chewable composition includes a delivery system for delivering dietary supplements and/or pharmaceutical compounds to a user's body. The delivery system includes a delivery vehicle in the form of a gummy candy, and a dietary supplement and/or a pharmaceutical compound as active ingredients of the gummy candy. The gummy candy may include a binding agent having thermo-irreversible characteristics, a sweetener, natural flavors and colors, and a polishing agent. For example, in one implementation, the gummy candy may include starch, sucrose, corn syrup, citric acid, lactic acid, natural colors, natural flavors, fractionated coconut oil, and carnauba wax.

The active ingredients may include an over-the-counter (OTC) drug or a prescription drug to provide a desired effect on the user. In addition to OTC or prescription drugs, the active ingredients may also include nutraceuticals (i.e., extracts of food purported to have a medicinal effect on human health) such as botanical and herbal extracts and antioxidants, or any combination of food supplements such as vitamins, minerals, soluble and insoluble fibers, herbs, plants, amino acids, probiotics, prebiotics, and digestive enzymes.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 shows a flow diagram of an example of a method of manufacturing a chewable supplement of the present invention.

FIG. 2 shows a flow diagram of an example of a method for incorporating a pharmaceutical compound into a delivery vehicle of a delivery system of the present invention.

### DETAILED DESCRIPTION

The present invention relates to a chewable candy, in particular, a heat resistant delivery system that is chewy or gummy-like and designed to enhance the delivery of nutritional supplements and pharmaceutical compounds. The delivery system includes a primary active ingredient to provide a desired effect, and a chewable delivery vehicle to contain the active ingredient for delivery.

In one implementation, the active ingredient of the present invention may include any combination of vitamins, minerals, antioxidants, soluble and insoluble fibers, herbs, plants, amino acids, probiotics, prebiotics, fatty acids, digestive enzymes, or any other ingredients digested to promote the heath and well-being of a person.

In other implementations, the active ingredient may include a pharmaceutical compound. For example, in one implementation, the pharmaceutical compound may include an OTC drug to treat symptoms of common illnesses. Such OTC drugs may include Benadryl®, Sudafed®, Claritin®, Maalox®, Mylanta®, Turns®, Pepcid® AC, Monistat®, Ex-Lax®, Imodium® A.D., Robitussin®, Chloraseptic®, Thera-flu®, Alka-Seltzer, Motrin®, Dramamine®, and the like, in liquid, powder, or extract form. In another implementation, the pharmaceutical compound may include a prescription drug. Such prescription drugs may include Lipitor®, Singulair®, Lexapro, Plavix®, Morphine, Hydrocodone (Vicodin®), Demerol®, Codeine, Diazepam (Valium®), Penicillin, Prevacid®, Allegra-D®, Celebrex®, Crestor®, Cialis®, Valtrex®, Ambien CR®, Viagra®, Flomax®, Prozac®, and the like, in liquid, powder, or extract form. In these implementations, in addition to an active pharmaceutical ingredient, the active ingredients of the delivery system may also include a combination of dietary supplements. The inclusion of dietary supplements with a pharmaceutical compound will depend in part on the supplements' compatibility with the pharmaceutical compound.

As for the dosage, pharmaceutical compounds are generally expressed in terms of grams or milligrams, but may also be expressed in active units, or international unit (IU). As used herein, a “pharmaceutical compound” or “drug” shall include, but is not limited to, any drug, hormone, peptide, nucleotide, antibody, or other chemical or biological substances used in the treatment or prevention of disease or illness, or substances which affect the structure or function of the body.

The active ingredient(s) are delivered in a delivery vehicle that is palatable and easy to swallow. In one implementation, the delivery vehicle may be a gummy candy to facilitate ingesting all of the active ingredients. The delivery vehicle may include a binder—having thermo-irreversible chemical characteristics, sweeteners, coloring, and flavoring.

As used herein, a “thermo-irreversible” binding agent refers to a gel or other compound that, after it solidifies, does not melt and retains its shape and integrity when heated. As used herein, “thermo-irreversible” binding agents may also include gels or other compounds that are thermally reversible, but their melting temperature is so high that they don't melt in practice.

As used herein, the term “melting temperature” refers to the temperature or temperature range over which a gel or compound softens or flows.

### Manufacturing of Delivery System

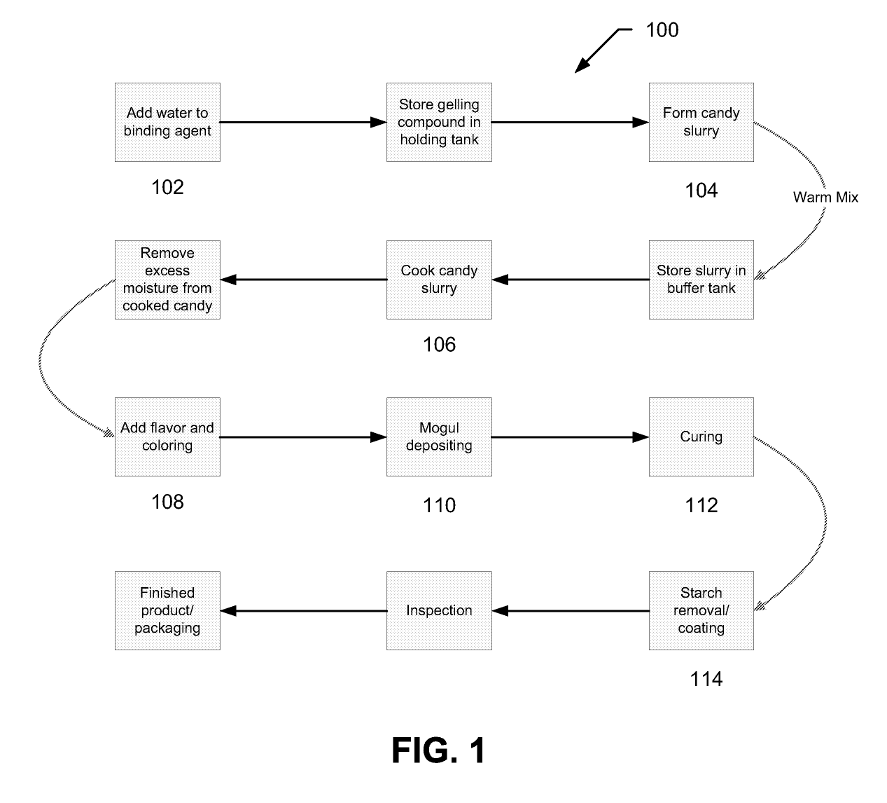

Turning now to FIG. 1, a method 100 for manufacturing a heat resistant delivery system of the present invention is disclosed. In general, the method of Manufacturing involves three main phases: (i) compounding (i.e., mixing) and storing; (ii) batching and cooking; and (iii) depositing and curing.

The first phase of compounding and storing begins with step 110, where water and a binding agent are mixed in a mixing tank to form a gelling compound. In one implementation, the mixing tank may include a 1,000 gallon stainless steel planetary mixer, a scrape surface mixer, a holding tank with an agitator, or any other suitable mixer. During production, water and the binding agent are continuously mixed in the mixing tank and the gelling compound is continuously turned in the tank by an agitator to keep the binding agent suspended in water (i.e., to prevent the binding agent from settling on the bottom of the mixing tank). In one implementation, approximately 6,000 lbs to 8,000 lbs of gelling compound may be produced in 8 hours.

The gelling compound may include cold, warm, or hot water. However, warm or hot water may be used to reduce the hydration time of the gelling compound. For example, about 250 lbs of gelatin mixed with about 250 lbs of warm water may reach a homogenous mixture in about 10 minutes. The hydration rate of the gelling compound may also vary according to the speed of the agitator. As used herein, “hydration time” or “hydration rate” refers to the time it takes water to hydrate the binding agent.

As used herein, the term “homogeneous” refers to a composition, solution or mixture whose elements are substantially uniformly dispersed in each other. For example, a homogenous composition may include two or more compounds or elements which are substantially uniformly dispersed within each other.

The binding agent may include food starch, carrageenans, or any other suitable binder having thermo-irreversible characteristics, or combination thereof. For example, the starch ingredients may include corn starch, rice starch, potato starch, starch derivatives, and the like.

Examples of carrageenan ingredients may include kappa (κ) carrageenans or lambda (λ) carrageeans. Carrageenan gels have a melting temperature and a setting point which is dependent on the concentration of potassium ions. At low concentrations of potassium ions, carageenan gels have a low melting temperature and setting point, but with increasing concentrations of potassium ions, the carageenan gel melting temperature and setting points increases. As used herein, the “setting point” refers to the temperature or temperature range in which a gel or other compound transitions from a viscous (liquid) gel to a solid gel.

In some implementations, a thermoreversible binding agent (i.e., a gelling compound that typically melts when heated) may be combined with a thermo-reversible binding agent to produce a heat resistant delivery vehicle in accordance with the present invention. For example, the gelling compound may include a combination of starch and gelatin, or starch and pectin. Depending on the binding agent used, the gelling compound may include, for example only, one of the following formulations:

TABLE A
GELLING COMPOUND FORMULA

| Binding | Binding Agent | Water |
|---|---|---|
| Agent | (% by weight) | (% by weight) |
| starch | 7%-10% | 90%-93% |
| pectin/starch | 8%-10% (1%-2% pectin/7%-8% starch) | 90%-92% |
| pectin/starch | 7%-9% (1%-2% pectin/6%-7% starch) | 93%-91% |
| carrageenan | 2%-5% | 95%-98% |

In one implementation, a buffer, such as hydroxides, carbonates, citrates and phosphates, mixtures thereof and their salts (e.g., sodium bisulfate or sodium citrate), may be mixed into the gelling compound to regulate the pH of the mixture. In one implementation, the gelling compound may contain approximately 0.01 to 0.03% buffer by weight, or any other suitable amount. The pH of the mixing tank may be adjusted to a range from about 3.2 to about 4.0 to provide adequate gelation and to ensure that the gelling compound does not become unstable (i.e., acidic) during mixing. In implementations where the binding agent includes starch, a buffer may not be necessary to balance the pH of the compound because starch is a stable organic compound.

At step 112, the gelling compound may be filtered through a fine mesh, to remove particulates in the compound, and stored in a holding tank. In one implementation, about 140 lbs to 190 lbs of gelling compound may be delivered from the mixing tank to the holding tank every 5 to 10 minutes. The filter may be a 0.034 inch stainless steel basket strainer and the holding tank may be a 1,500 gallon stainless steel tank. In some implementations, the holding tank may include a moderate agitator (e.g., mixing blades) to mix the compound and prevent the binding agent from settling on the bottom of the holding tank during storage.

From the holding tank, approximately 125 lbs to 185 lbs of gelling compound may be delivered to a mixing vessel at step 114, every 5 to 10 minutes, for example. In one implementation, the mixing vessel may be a 5,000 gallon stainless steel planetary mixer. In other implementations, the mixing vessel may be a scrape surface mixer, a holding tank with an agitator, or any other type of suitable mixer.

In the mixing vessel, water, additives, supplements, and an active ingredient may be added to the gelling compound to form a candy slurry mixture. For example, the additives may include sodium citrate, sweeteners such as sugar (also referred to herein as sucrose or natural cane juice) and/or syrup (e.g., corn, glucose, rice, tapioca), and corn starch, in liquid and/or powdered form. In one implementation, the supplements and/or active ingredient may include vitamins, minerals, herbs, plants, amino acids, probiotics, prebiotics, fatty acids, enzymes or any other supplements digested to promote the heath and well-being of a person. The supplements may include, but not be limited to, any of the following:

Vitamin B1 (Thiamine)

Vitamin B2 (Riboflavin)

Vitamin B3 (Niacinamide)

Vitamin B5 (Pantothenic Acid)

Vitamin B6 (Pyridoxine HCL)

Vitamin B12

Biotin

Folic Acid

Vitamin C (Ascorbic Acid/Activated C)

Calcium

Carotine

Chromium

Choline

Copper

Magnesium

Zinc

Protein

Pomegranate

Inositol

Vitamin D (Cholecalciferol)

Vitamin E (Acetate)

Gingseng

Iron

Vitamin K (Phytonadione)

St. John's Wort

Omega fatty acids

The above list of raw materials are not exhaustive, but are provided for illustrative purposes only. The length of a list of all available supplements that may be utilized in the chewable supplement of the invention is too lengthy to provide.

In one implementation, the candy slurry mixture may contain approximately 70% to 85% sweetener by weight, while the remaining approximately 15% to 30% of the slurry (by weight) may contain the gelling compound and additives. More particularly, the slurry may contain approximately 19% water, 2% sodium citrate, 30% natural cane juice, 45% corn syrup, 2% supplements, and 2% primary active ingredient by weight. In most implementations, the candy slurry may reach a homogeneous mixture in about 5 to 10 minutes.

The ingredients described above and their compositions are provided by way of example only. Without departing from the spirit and scope of the present invention, the ingredients and the composition of the candy slurry may vary based on the type of formulation desired.

Prior to production, the sugar and syrup additives may be stored in bulk tanks. In one implementation, the syrup may be stored in a holding tank at a temperature of approximately 75° F. In the holding tank, the syrup may be irradiated by ultraviolet light to remove any contaminants in the syrup. The syrup may include high fructose corn syrup (e.g., HFCS-42, HFCS-55, or HFCS-62), glucose syrup, rice syrup, tapioca syrup, or any other suitable liquid sweetener or combination thereof. During production, the syrup may be administered to the mixing vessel manually or by automation.

Similarly, prior to production, sugar in granular form may be stored in a holding tank. During production, sugar may be fed through an automated feed system that filters the sugar to remove sediments, weighs the sugar, and delivers a desired quantity of sugar to the mixing vessel. In other implementations, sugar may be added to the mixing vessel manually.

Turning back to FIG. 1, from the mixing vessel, the candy slurry is processed through a magnetic device, which removes particulates from the slurry, and stored in a storage buffer tank at step 116. In one implementation, the magnetic device may be a finger magnet or any other suitable magnetic device, and the storage tank may be a 5,000 gallon stainless steel industrial holding tank. In other implementations, the holding tank may include a moderate agitator to suspend the active ingredients in the candy slurry. Prior to reaching the storage buffer tank, the candy slurry may be heated through a series of heat exchangers to a temperature of approximately 150° F. to 180° F.

In one implementation, the storage buffer tank may receive the candy slurry mixture from the mixing vessel at a mass flow rate of approximately 15 lbs/s to 20 lbs/s, and maintain the slurry at a temperature of approximately 150° F. to 200° F. Simultaneously, the warm candy slurry may be continuously fed from the storage buffer tank to a static cooker at mass flow rate of approximately 10 lbs/s to 15 lbs/s, by way of example only.

In the next phase of batching and cooking, at step 118 the candy slurry mix is received by the static cooker and cooked at a temperature of approximately 220° F. to 260° F. for approximately 30 sec. to 60 sec., until the slurry is gelatinized (i.e., dehydrated). In one implementation, the static cooker may be a 2,500 gallon high pressure steam jacketed kettle, a vacuum pressure cooker, or any other suitable cooker. In the static cooker, moisture is evaporated out of the candy slurry as the slurry is boiled to a temperature of approximately 250° F. After about a minute of boiling, the slurry may consist of about a 65 to 75 brix solution.

As used herein, the term “brix” refers to the dissolved sugar-to-water ratio of a liquid or gel. For example, as described above, the slurry mixture may consist of approximately 65 grams to 75 grains of sugar and approximately 25 grains to 35 grams of water per 100 grams of solution.

After the candy slurry is cooked, a vacuum is applied to the candy at step 120. In one implementation, the pressure cooker may include a vacuum apparatus. In another implementation, the cooked candy may be delivered to an industrial vacuum chamber or any other suitable enclosure.

At the vacuum step 120, moisture is drawn from the cooked candy by suction pressure. In one implementation, a vacuum of approximately 40 psi to 50 psi is applied to the candy stock for approximately 15 sec. to 30 sec. may draw out approximately 2% to 5% water by weight. However, the pressure of the vacuum and the vacuum rate will vary according to the capabilities and size of the vacuum apparatus. At this juncture, the cooked candy may have a brix of approximately 67 to 80, and a pH of approximately 2.8 to 4.0, for example.

From the vacuum, the cooked candy is filtered through a strainer into a trough-like apparatus, commonly known as a dosier. In other implementations, the process of adding flavoring and coloring to the cooked candy, as described below, may be performed by an apparatus incorporated within a starch molding machine.

At this point in the manufacturing process, the cooked candy mainly consists of a clear gelatinized composition. To obtain a desired color and taste, coloring and flavoring may now be added to the cooked candy.

At step 122, the cooked candy may be passed through the dosier. In the dosier, water, flavoring, coloring, and food grade acid may be added to the cooked candy to enhance the candy's taste. For example, flavoring such as artificial and/or natural flavoring (e.g., fruit concentrate) may be added to the cooked candy to give the candy a desired flavor. To balance the flavor, food grade acid may be added to the cooked candy. Such food acids may include citric acid, malic acid, lactic acid, adipic acid, fumaric acid, tartaric acid, or any other suitable food acid or combinations thereof. In one implementation, the flavoring, coloring, and acid may be continuously added to (e.g., dripped on) the cooked candy as the candy moves through the dosier to the mogul machine.

The amount of flavoring, coloring, and acid administered to the cooked candy may vary according the volume of cooked candy passing through the dosier and the desired candy formulation. For example, in one implementation, approximately 1% to 2% flavoring by weight and approximately 0.01% to 0.03% acid by weight may be added to the cooked candy composition. However, the amount of acid and flavoring added to the cooked candy formulation must be balanced to insure that the cooked candy will taste good. So, depending on the formulation, more flavoring and less acid may need to be added to the cooked candy for bitter formulations. In some instances, only food acid instead of flavoring may be added the cooked candy.

In addition to food acid, coloring and titanium dioxide may be added to the cooked candy formulation in the dosier. Coloring may be added to give the candy a desired color or colors. Coloring may include natural coloring such as black carrot, annatto, tumeric, and purple berry concentrate, or artificial coloring such as yellow 5, red 3, and blue 1, or any combination thereof.

Titanium dioxide may be added to the candy to provide sheen. Titanium dioxide may also stabilize the cooked candy formulation so the coloring does not bleed when it is handled, packaged, or stored.

In the final phase of depositing and curing, after the cooked candy is passed through the dosier, the candy may be sent to a starch molding machine at step 130. In one implementation, the starch molding machine may include a mogul machine (simply referred to as a “mogul”). A mogul is a starch molding machine that automatically performs the multiple tasks involved in making gummy candy.

Gummy candy is produced in the mogul by a continuous process. At the start the process, the cooked candy, or gummy stock, is deposited by depositors (e.g., filling nozzles) onto starch lined trays (“mogul boards”) that allow the cooked candy to firm and take on the shape of the tray mold, to produce a series of shaped gummy candies. In one implementation, the depositors are timed to automatically deliver the exact amount of candy needed to fill the trays as the mogul boards are passed under the depositors. In other implementations, the coloring, flavoring, and acids added to the cooked gummy candy at step 122, may be added to the candy in the depositor.

A mogul is called a starch molding machine because starch is a main component of the machine. In this machine, starch has three primary purposes. First, it prevents the gummy candy stock from sticking to the mogul boards, which allows for easy removal and handling. Second, starch holds the gummy candy in place during the drying, cooling, and setting processes. Finally, starch absorbs moisture from the candies, giving them the proper texture.

In some cases, the starch used to coat the mogul boards may include re-used starch that is sifted and dried in a starch dryer, and then cooled in a starch cooler. The cooled starch is sifted a second time and placed in the mogul where it is recirculated through the same process. The recirculated starch may then be sprayed evenly on the mogul board. The cooked candy may then be deposited onto mogul boards coated with the recirculated starch.

After the cooked candy is deposited onto the mogul boards, the mogul boards may be stacked, then removed from the stack (one-by-one) by a conveyor belt, and finally placed in a temperature and humidity controlled curing room, where the candy sits and cools (i.e., is cured) for approximately 24 hours to 48 hours (step 132). However, the curing time for the cooked candy may vary based on binding agent and the temperature and humidity of the curing room. Proper curing time is necessary to solidify, or set the gummy product to ensure ease of packaging without breakage and proper yield. In one implementation, the candy may be cured in a curing room with approximately 15% to 25% humidity.

After curing, the gummy candies, firmed and having proper texture, may be moved to a section of the mogul called the starch buck. In the starch buck, the mogul boards are inverted and the gummy candy is dumped into a tumbler machine at step 134. In one implementation, the tumbler may include a 2,000 gallon rotating drum or, in implementations, a vibrating metal sieve. In the tumbler, the gummies may be tumbled together to remove any excess starch that adheres to the gummy candies. Once the starch is removed, the gummies may become sticky, so the gummies may be coated with a polishing compound to prevent the cooked candies from sticking together. Depending on the desired finished product or preferences, the gummies may be polished with fractionated coconut oil, linseed oil, sunflower oil, bees wax, carnauba wax, mineral oil, or any other suitable food grade oil or combination thereof. In other implementations, the gummies may be sanded with sugar or a sugar substitute in a drum.

After the gummies are coated, they may be placed on a cooling belt (e.g., a conveyor belt) and transported to an inspection station at step 136. At step 136, the gummy candies are placed on an inspection belt where the candies are inspected for food safety and proper organoleptic effects. For example, on the inspection belt the gummy candies may be passed by a detector or x-ray to insure that no particulate or other foreign material has been deposited into the candy during the depositing stage.

Moving on to step 138, once the candy passes inspection, the finished gummy product is packaged for distribution.

The disclosure above only describes one implementation of a method of manufacturing a delivery system of the present invention. Other methods and implementations may be used to manufacture delivery systems in accordance with the present invention.

### Addition of Pharmaceutical Compound

Pharmaceutical compounds may be incorporated into a delivery system of the present invention by one of three methods: (i) as a liquid or solid prior to cooking the gummy composition; (ii) by encapsulation; or (iii) in liquid or extract form after the gummy composition has been cooked. The manner in which a pharmaceutical is incorporated into the delivery system depends on the heat sensitivity and chemical composition of the drug.

For example, under the first method, a drug may be added to the gelling compound at step 114 (FIG. 1), during the mixing and storing phase. In one implementation, the drug may be poured into the mixing vessel in solid, powdered, extract, or liquid form.

Because many pharmaceutical compounds are destroyed or degraded when exposed to heat, this method may not be effective for heat-sensitive drugs. For instance, in the mixing phase of the gummy manufacturing process, the gelling compound may be heated to a temperature of 185° F. Thus, the chemical structure of a drug incorporated into the delivery system under this method must be able to withstand temperatures in excess of 200° F.

For heat-sensitive drugs, such as lorazepam, the second method of encapsulation may be applied. Under this second method, the drug may be encapsulated before it is added to the gelling compound at step 114 (FIG. 1). This method may be most effective for drugs, in solid or powdered form, that are moderately resistant to heat.

Prior to encapsulation, the drug may be pulverized to within a discrete particle size ranging from approximately 10 microns to 300 microns; the smaller the particle size, the more effective the encapsulation. Because the drug is encapsulated, the drug release and absorption capabilities of the delivery system may be controlled depending on the effectiveness of the encapsulation. For example, encapsulation may prevent early release of the drug to the user's system.

In one implementation, a solvent system containing a filming agent may be mixed with the drug particles and blended at slow speed in a planetary mixer. The solvent may be water or ethanol and the filming agent may be ethylcellulose, gelatin, a water-soluble plasticizer (e.g., glycerin or xylitol), or any other suitable composition. The filming agent solution may be slowly added to the drug particles so that enough individual particles will adhere together to form larger granules having a size of approximately 300 to 500 microns. The degree of encapsulation may vary depending upon the number of layers of filming agent solution applied. In one implementation, the film coating may have a thickness of about 1 micron or less. There exist various standard pharmaceutical coating techniques that are suitable for use with this invention, depending on the filming agent, type of active ingredient that is to be coated, and the drug release objective, such as immediate release versus sustained release.

Under the third method, heat sensitive drugs may be added to the cooked candy at step 122, during the flavoring and coloring phase. In one implementation, a drug in liquid or extract form may be added to the cooked candy in the dosier with the coloring and/or flavoring. While in other implementations the drug may be added in solid or powdered form, drugs in the form of liquid or extracts are preferred at this stage of the manufacturing process because liquids and extracts are better incorporated into the cooked candy.

The amount of flavoring added to the cooked candy will vary depending upon the desired flavor and amount of pharmaceuticals added to the gelling compound. Some pharmaceuticals will require differing amounts of flavor, sweetener, color, and food acid to produce a desirable tasting chewable drug. For example, to mask the flavor of a particular drug, a flavoring agent such as strawberry flavor or cherry flavor may be added to the mixture. The additional flavor would be adjusted based upon the drug. For extra bitter drugs, a flavor masking flavor compound from flavor houses may also be utilized.

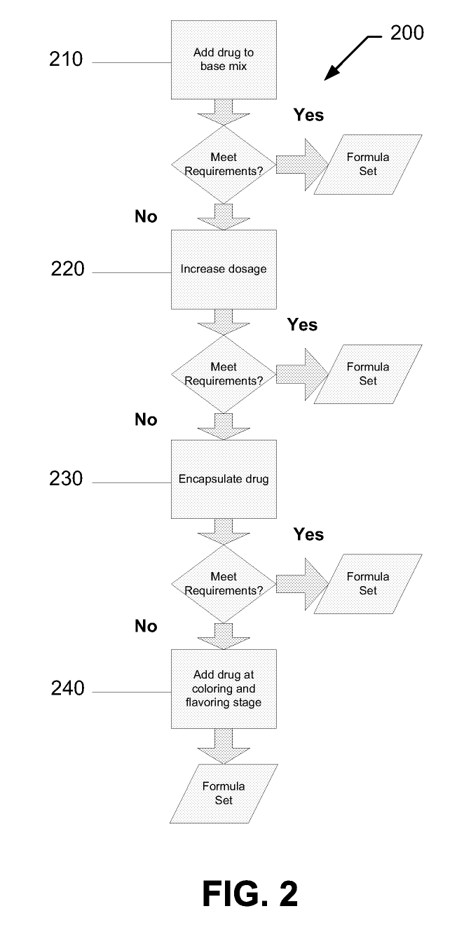

Turning now to FIG. 2, one implementation of a method 200 of incorporating a pharmaceutical into the delivery system of the invention is described. According to this method, the first step (step 210) is to prepare a test batch of gummy candies adding the drug to the gelling compound in the mixing vessel, at step 114 (FIG. 1) of the manufacturing process. After the gummy candy is cooked, cooled, and cured, the candies may be inspected and tested at step 136 (FIG. 1) to validate that the drug composition of the candies meet the desired label requirements (i.e., meet the dosage printed on the product label). If the drug composition is validated, then the chemical formulation of the finished gummy product is set and the gummy candies may be mass produced and packaged using the first method of incorporation described above.

If the drug composition is not validated (i.e., the drug composition breaks down because the drug is heat sensitive), a second test batch may be produced and tested. This time, the dosage of the drug added to the gelling compound at step 114 (FIG. 1) may be increased to compensate for the drugs broken down during the cooking phase (step 220). For example, if 100 mg of aspirin is added to the gelling compound in the mixing weigh vessel to produce a 75 mg drug, but only 50 mg of aspirin is measured in the finished product, then 150 mg of aspirin may be added to the gelling compound in the mixing weigh vessel during the second production to compensate for the 25 mg of aspirin dissipated during the manufacturing process.

Once tested, if the drug composition is validated, then the chemical formulation of the finished gummy product is set and the gummy candies may be mass produced and packaged using the first method of incorporation described above. However, if second batch does not meet the label requirements, the drug may need to be encapsulated or added at a different stage of the manufacturing process.

If encapsulation is required, then a third test batch of gummy candies may be produced (step 230). In this step, the encapsulated drug may be added to the gelling compound in the mixing vessel, at step 114 (FIG. 1) of the manufacturing process, and the gummy candies are tested once again. If the gummy candies meet the label requirements, then the chemical formulation will be set (with an encapsulated drug), and the gummies may be mass produced and packaged using the second method of incorporation described above.

If the encapsulated gummy candies do not meet the label requirements, then the drug may need to be incorporated into the cooked candy as an oil, extract, or liquid in the flavoring and coloring phase of the manufacturing process (step 240). In this step, a fourth test batch may be produced where a liquid or extract drug may be added to the cooked candy with the coloring and flavoring at step 122 of the manufacturing process. After the gummy candies are produced, the batch may be tested once again to validate the drug composition of the candies. If the drug composition is validated, then the chemical formulation of the gummy product is set and the gummy candies may be mass produced and packaged using the third method of incorporation described above. If the third batch does not meet the label requirements, the dosage of the liquid or extract may need to be adjusted accordingly at step 122 (FIG. 1).

The process described above may only apply to drugs generally sold in granule, solid, or powder form. Any drugs generally sold in oil, liquid, or extract form may be automatically added to the cooked candy in the flavoring and coloring phase of the manufacturing process.

Delivery systems of the present invention not only make drugs palatable, the chewy consistency of the delivery system allows drugs to be easily digested by users of all ages, particularly, those users who have problems swallowing pills. In addition, the formulation of the delivery system enhances the absorption of drugs into the blood stream. Also, for users who cannot digest large drug dosages, the chewable drugs of the present invention will allow these users to administer smaller drug dosages at one time (i.e., the user can take five 10 mg gummies instead of taking one 50 mg drug dosage), which will allow the body to quickly absorb the drug.

### EXAMPLES

The following examples describe particular formulations and concentrations thereof for preparing chewable supplements of the present invention. Chewable supplements of the present invention may include non-organic and/or organic compositions. For example, in one implementation, the chewable supplement may include a non-organic or an organic gummy candy. While the process of manufacturing a non-organic gummy and an organic gummy are similar, as described above, the formulations for the two systems differ, as explained in more detail below.

### Non-Organic Drug

In one implementation, the delivery system of the present invention may include a non-organic gummy. For example, a 50 mg non-organic chewable aspirin in accordance with the present invention may be prepared using the following formula:

TABLE B
NON-ORGANIC GUMMY FORMULA

| Ingredients | Content (by Weight) |
|---|---|
| Water | 6.0% |
| Lactic acid | 1.0% |
| Citric Acid | 1.0% |
| Sucrose | 35.3% |
| Corn Syrup | 45.0% |
| Starch | 9.0% |
| Aspirin (50 mg) | 0.2% |
| Flavoring (natural/artificial) | 1.5% |
| Colorant (natural/artificial) | 1.0% |

In this example, about 91 lbs of warm water may be mixed with about 9 lbs of starch compound in the mixing tank to form 100 lbs of gelling compound having a homogeneous 91/9 blend of water and starch. In one implementation, the starch ingredient may include corn starch, rice starch, modified starches, or any other suitable starch ingredient.

In the mixing weigh vessel, the gelling compound may be mixed with about 6 lbs of water, 35.3 lbs of sucrose, and 45 lbs of corn syrup to form the candy slurry mixture. Because aspirin is not a heat sensitive drug, about 0.15 lbs to 0.2 lbs of aspirin may be added to the candy slurry at step 114 (FIG. 1).

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of strawberry flavoring by weight and about 1% of red cabbage coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### Organic Vitamin

In another implementation, the delivery system of the present invention may include an organic gummy. To create an organic gummy, the ingredients used to form the drug must meet the requirements for organic certification. These ingredients may include, but not be limited to, organic evaporated cane juice, organic tapioca syrup, organic grape juice, citric acid, lactic acid, sodium citrate, natural color (e.g., black carrot juice concentrate, annatto, turmeric, purple berry concentrate) and natural flavor (e.g., strawberry, orange, pineapple, grape), and a proprietary blend of vitamins, minerals and other functional ingredients.

For example, a 220 mg organic chewable multi-vitamin, in accordance with the present invention, may be prepared using the following formula:

TABLE C
ORGANIC GUMMY FORMULA

| Ingredients | Content (by Weight) |
|---|---|
| Water | 7.5% |
| Lactic acid | 1.0% |
| Citric acid | 1.0% |
| Organic evaporated cane juice | 33.0% |
| Organic tapioca syrup | 44.0% |
| Starch/pectin composition | 8.0% |
| Multi-vitamin blend (220 mg) | 3.0% |
| Natural flavoring | 1.5% |
| Natural colorant | 1.0% |

In this example, about 92 lbs of warm water may be mixed with a combination of about 3 lbs of starch and pectin in the mixing tank, to form 100 lbs of gelling compound having a homogeneous 92/8 blend of water and starch/pectin. While a starch/pectin composition is described as the binding agent in this specific example, other combinations, for example, a starch/organic gelatin or a starch/carrageenan composition may also be used as the binding agent in other implementations. To stabilize the gelling compound, about 0.1% to 10% of sodium bisulfate by weight may be added to the compound to reduce its pH to about 3.5.

In the mixing weigh vessel, the gelling compound may be mixed with about 6 lbs of water, 33 lbs of organic evaporated cane juice, and 44 lbs of organic tapioca syrup to form the candy slurry mixture. In addition to sweeteners, about 2.5 lbs to 3 lbs of multi-vitamin blend may be added to the candy slurry at step 114 (FIG. 1). In one implementation, the multi-vitamin blend may include approximately 2500 IU of Vitamin A, 2 mg of Vitamin B-6, 6 mcg of Vitamin B-12, 60 mg of Vitamin C, 400 IU of Vitamin D, 15 mg of Magnesium, 15 mcg of Choline, 15 mg of Zinc, 20 mg of Calcium, 150 mcg of Iodine, and 15 mcg of Inositol. To stabilize the candy slurry mixture, about 0.1% sodium citrate by weight may also be added to the slurry to maintain its pH at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of orange and cherry flavoring by weight and about 1% of annatto and turmeric coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

### Carrageenan Vitamin

In another implementation, the delivery system of the present invention may include a carrageenan-based gummy. For example, a 220 mg carrageenan-based chewable multi-vitamin, in accordance with the present invention, may be prepared using the following formula:

TABLE D
CARRAGEENAN GUMMY FORMULA

| Ingredients | Content (by Weight) |
|---|---|
| Water | 9.0% |
| Lactic acid | 1.0% |
| Citric acid | 1.0% |
| Sucrose | 35.0% |
| Corn Syrup | 46.0% |
| λ-carrageenan | 2.5% |
| Multi-vitamin blend (220 mg) | 3.0% |
| Natural flavoring | 1.5% |
| Natural colorant | 1.0% |

In this example, about 96 lbs of warm water may be mixed with a combination of about 2.5 lbs of λ-carrageenan in the mixing tank, to form 100 lbs of gelling compound having a homogeneous 97.5/2.5 blend of water and carrageenan. To stabilize the gelling compound, about 0.1% to 10% of sodium bisulfate by weight may be added to the compound to reduce its pH to about 3.5.

In the mixing weigh vessel, the gelling compound may be mixed with about 6 lbs of water, 35 lbs of organic evaporated cane juice, and 46 lbs of organic tapioca syrup to form the candy slurry. In addition to sweeteners, about 2.5 lbs to 3 lbs of multi-vitamin blend may be added to the candy slurry at step 114 (FIG. 1). In one implementation, the multi-vitamin blend may include approximately 2500 IU of Vitamin A, 2 mg of Vitamin B-6, 6 mcg of Vitamin B-12, 60 mg of Vitamin C, 400 IU of Vitamin D, 15 mg of Magnesium, 15 mcg of Choline, 15 mg of Zinc, 20 mg of Calcium, 150 mcg of Iodine, and 15 mcg of Inositol. To stabilize the candy slurry mixture, about 0.1% sodium citrate by weight may also be added to the slurry to maintain its pH at about 3.0 to 3.5.

Next, the candy slurry may be heated to a temperature of about 180° F. prior to being passed through the storage buffer tank, to the static cooker. In the static cooker, the candy slurry may be heated to a temperature of about 240° F. to 245° F., dehydrating the slurry to a brix of about 78.

After the candy is cooked, the cooked candy is sent to the vacuum, where the candy may be further dehydrated to a brix of about 80. After leaving the vacuum, the cooked candy is placed in the dosier where about 1.5% of orange and cherry flavoring by weight and about 1% of annatto and turmeric coloring by weight may be added to the cooked candy. To balance the flavoring, about 0.1% citric acid by weight and about 0.1% lactic acid by weight may be added to the cooked candy.

After adding the flavoring and coloring, the cooked candy may be deposited into the mogul machine and then cured. After the candies are cured, they may be added to a tumbling drum to break off any starch that may be remaining on the candies. As the candies are being tumbled, about 1% fractionated coconut oil by weight and about 1% carnauba wax by weight may be poured into the drum to coat the candies to prevent them from sticking together.

After the candies are coated, they may be inspected to validate that the finished product meets the label requirements, and then packaged.

In accordance with the teachings of the present invention, starch-based gummies provide an additional benefit over traditional gelatin-based gummies, or any other candy made from a thermoreversible gelling agent. In particular, because gelatin liquefies when heat is applied, gelatin-based gummies frequently melt when they are exposed to high temperatures during storage and transport. But starch is more stable than gelatin in high temperatures. This is because the semi-crystalline structure of starches do not fully recover once a starch is gelatinized (i.e., becomes a gel when cooked in water) and then cooled, so the starch becomes a thickened paste. If additional heat is applied to the thickened paste, the starch will not liquefy since the starch granules swell and burst during the gelatinization process. Thus, starch-based gummies may retain their gummy shape under high temperature without melting. This is ideal for gummies that are exposed to high temperatures during storage and transport.

The examples provided above are for illustrative purposes only. Formulations for chewable drugs of the present invention may vary based on the desired dosage of the active pharmaceutical ingredients and the amount of additives, sweeteners, and coloring added to the drug composition. Thus, testing will be required to arrive at a suitable composition for each chewable drug.

While implementations of the invention have been described with reference to a gummy delivery system, the invention is not limited to this application and may be readily used for any chewable composition that includes a thermo-irreversible composition. For example, implementations of the invention may also be employed in organic, vegetarian or non-vegetarian, tablets, capsules, or solid candies. The present invention may also apply to other forms of candies such as jelly beans or caramel-based candies. Further, while the dimensions of the holding and mixing vessels are provided herein by way of example only, the actual dimensions of these vessels may vary based on the amount of gelling compound and candy slurry produced in a given time period (e.g., per day).

The foregoing description of implementations has been presented for purposes of illustration and description. It is not exhaustive and does not limit the claimed invention to the precise form disclosed. Modifications and variations are possible in light of the above description or may be acquired from practicing the invention. The claims and their equivalents define the scope of the invention.

What is claimed is:

1. A chewable composition comprising:
a binding agent having thermo-irreversible characteristics;
a sweetener; and
an active ingredient.

2. The chewable composition of claim 1 where the active ingredient is a dietary supplement.

3. The chewable composition of claim 2 where the dietary supplement includes any combination of vitamins, minerals, herbs, probiotics, prebiotics, fatty acids, digestive enzymes, or any other health promoting ingredient.

4. The chewable composition of claim 1 where the active ingredient is a pharmaceutical compound.

5. The chewable composition of claim 4 where the pharmaceutical compound is an over-the-counter drug.

6. The chewable composition of claim 4 where the pharmaceutical compound is a prescription drug.

7. The chewable composition of claim 4 further comprising any combination of vitamins, minerals, herbs, probiotics, prebiotics, fatty acids, digestive enzymes, or any other health promoting ingredient.

8. The chewable composition of claim 1 where the binding agent includes any combination of starch or carrageenan.

9. The chewable composition of claim 1 where the binding agent includes starch in combination with pectin, gelatin, or carrageenan.

10. The chewable composition of claim 1 where the binding agent includes carrageenan in combination with starch, pectin, or gelatin.

11. The chewable composition of claim 1 where the sweetener includes an organic compliant binding agent and sweetener.

12. The chewable composition of claim 11 where the composition qualifies as a composition capable of being certified as organic.

13. A heat resistant delivery system for health supplements comprising:
a chewy gummy candy that includes a binding agent having thermo-irreversible characteristics and a sweetener; and
an active ingredient incorporated into the candy.

14. The delivery system of claim 13 where the active ingredient is a dietary supplement.

15. The delivery system of claim 14 where the dietary supplement includes any combination of vitamins, minerals, herbs, probiotics, prebiotics, fatty acids, digestive enzymes, or any other health promoting ingredient.

16. The delivery system of claim 13 where the active ingredient is a pharmaceutical compound.

17. The delivery system of claim 16 where the pharmaceutical compound is an over-the-counter drug.

18. The delivery system of claim 16 where the pharmaceutical compound is a prescription drug.

19. The delivery system of claim 16 further comprising any combination of vitamins, minerals, herbs, probiotics, prebiotics, fatty acids, digestive enzymes, or any other health promoting ingredient.

20. The delivery system of claim 13 where the binding agent includes any combination of starch or carrageenan.

21. The delivery system of claim 13 where the binding agent includes starch in combination with pectin, gelatin, or carrageenan.

22. The delivery system of claim 13 where the binding agent includes carrageenan in combination with starch, pectin, or gelatin.

23. The delivery system of claim 13 where the sweetener includes an organic compliant binding agent and sweetener.

24. The delivery system of claim 23 where the composition qualifies as a composition capable of being certified as organic.
